(19) United States
(12) Patent Application Publication
(10) Pub. No.: US 2020/0397693 A1
(43) Pub. Date: Dec. 24, 2020

(54) COMPOSITIONS COMPRISING CANNABIS SATIVA OIL AND USES THEREOF

(71) Applicant: Hempvana, LLC, FairField, NJ (US)

(72) Inventor: Ajit Khubani, Saddle River, NJ (US)

(21) Appl. No.: 16/911,334

(22) Filed: Jun. 24, 2020

Related U.S. Application Data
(60) Provisional application No. 62/865,427, filed on Jun. 24, 2019.

(51) Int. Cl. A61K 9/06; A61K 31/133; A61K 31/216; A61K 9/00; A61K 47/10; A61K 47/46; A61K 47/44; A61K 47/34; A61K 47/32; A61K 47/22; A61K 47/12; A61K 47/14

(52) U.S. Cl. CPC A61K 9/06; A61K 31/133; A61K 31/216; A61K 9/0014; A61K 47/10; A61K 47/14; A61K 47/44; A61K 47/34; A61K 47/32; A61K 47/22; A61K 47/12; A61K 47/46

(57) ABSTRACT

The subject invention provides topical skin moisturizing compositions comprising Cannabis sativa oil and uses thereof.

### CROSS-REFERENCE TO RELATED APPLICATIONS

This application claims the benefit of U.S. Provisional Application No. 62/865,427, filed Jun. 24, 2019.

### FIELD OF THE INVENTION

The present invention relates to topical compositions comprising Cannabis sativa oil and a topical analgesic which may be used to provide enhanced pain relief.

### BACKGROUND OF THE INVENTION

Pain can be detrimental to qualify of life. There are many ways of treating pain. One method of alleviating pain is to apply a topical analgesic on the skin. Topical analgesics can be used to provide pain relief for various body pains, including back, neck, shoulders, feet, wrists and calves. Topical analgesics can also be used for arthritis, back, shoulder, joint and muscle pain. Topical analgesics are used to treat both acute pain and chronic pain.

One common topical analgesic is Trolamine Salicylate. It is an organic compound formed between triethanolamine and salicylic acid. It is used to treat aches and pains of the muscles and/or joints. There are several commercially available topical formulations with Trolamine Salicylate, including Aspercreme Cream, Myoflex Cream, and Sportscreme. Another common topical analgesic is Menthol. It is also used to treat aches and pains of the muscles and/or joints. There are several commercially available topical formulations with Menthol, including Icy Hot Arthritis Pain Relief, Tiger Balm Arthritis Rub, and Sarna Lotion.

Accordingly, there is a need for alternative topical formulations for treating pain. There is also a need for topical formulations with improved pain relief efficacy and moisturizing properties. The objective of this application is to provide alternative and improved topical compositions and methods for treating pain including Cannabis sativa oil in combination with a topical analgesic.

### SUMMARY OF THE INVENTION

The subject invention provides a topical skin moisturizing composition comprising an effective amount of Cannabis sativa oil in combination with a topical analgesic.

In an embodiment, the topical skin moisturizing composition includes Cannabis sativa oil in an effective amount from about 1% to about 90% by weight based on the total weight of the composition.

In another embodiment, the topical skin moisturizing composition includes at least one topical analgesic in an effective amount from about 1% to about 20% by weight based on the total weight of the composition.

In another embodiment, the Cannabis sativa oil is Cannabis sativa seed oil.

In another embodiment, the topical skin moisturizing composition includes one or more antioxidants and one or more colorants.

In another embodiment, the topical skin moisturizing composition is topically applied to the skin to alleviate pain and to moisturize the skin.

### BRIEF DESCRIPTION OF THE DRAWING

FIG. 1 shows sample labels for Example 1 and 2 of the present invention.

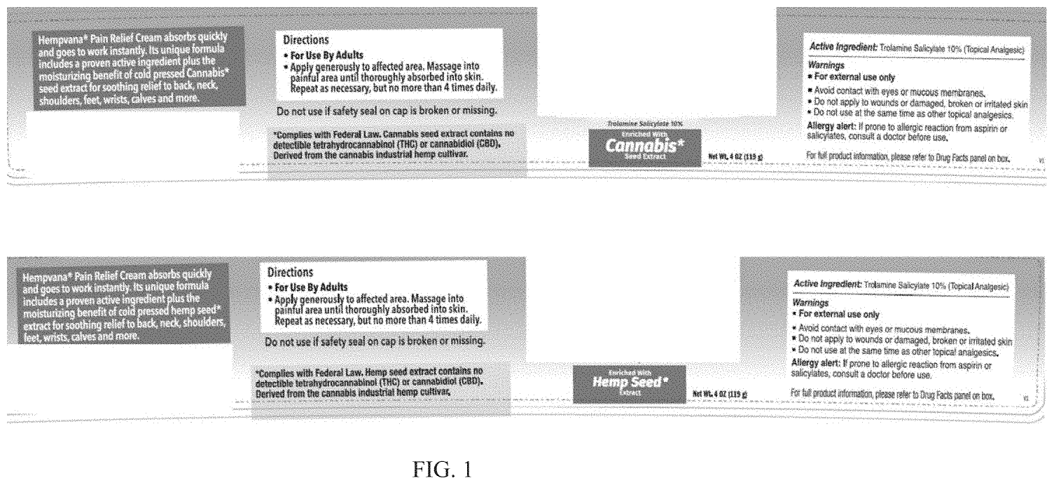

FIG. 2 shows sample labels for Example 1 and 2 of the present invention.

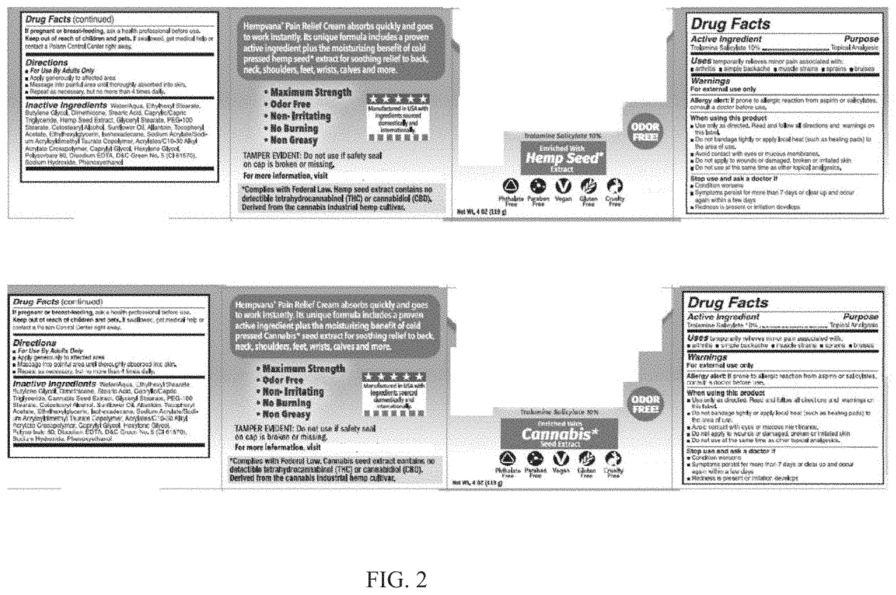

FIG. 3 shows sample labels for Example 3 of the present invention.

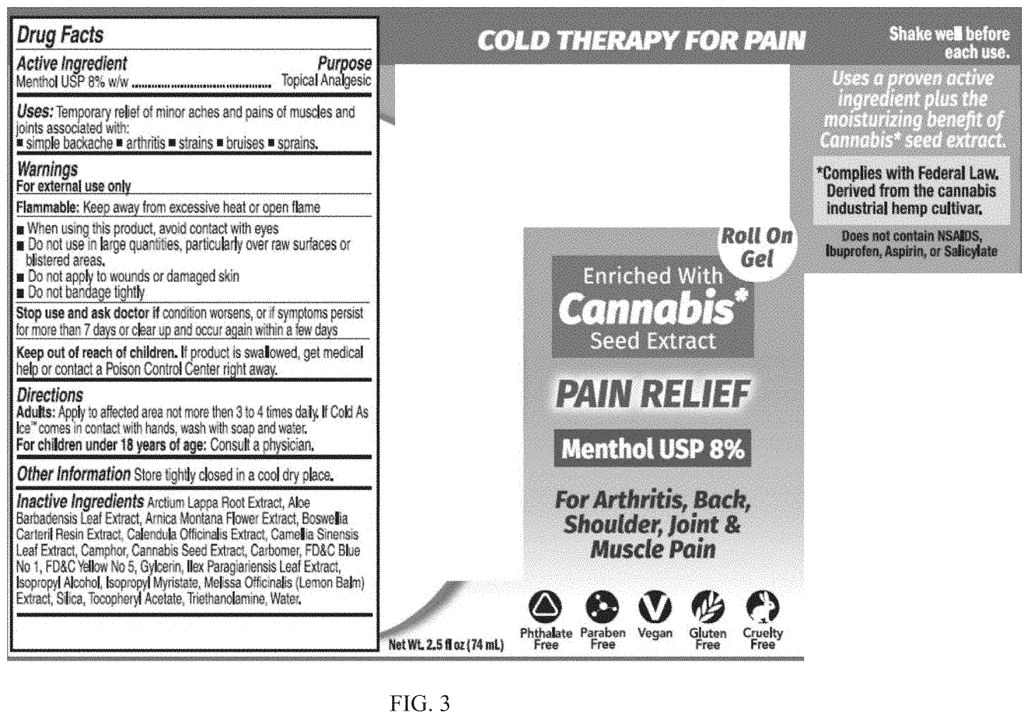

### DETAILED DESCRIPTION OF THE INVENTION

The subject invention provides a topical skin moisturizing composition comprising an effective amount of Cannabis sativa oil in combination with a topical analgesic. Although illustrative examples of one or more embodiments are provided below, the disclosure should not be limited to the illustrative examples below, but may be modified within the scope of the appended claims along with their full scope of equivalents. All composition related percentages disclosed herein are by weight based on the total weight of the composition.

An embodiment provides a topical skin moisturizing composition, including an effective amount of Cannabis sativa oil from about 1% to about 90% by weight based on the total weight of the composition. In another embodiment, the amount of Cannabis sativa oil in the composition is from about 1% to about 10% by weight based on the total weight of the composition. In a further embodiment, the amount of Cannabis sativa oil in the composition is from about 1% to about 5% by weight based on the total weight of the composition. In yet another embodiment, the amount of Cannabis sativa oil in the composition is from about 5% to about 10% by weight based on the total weight of the composition. In yet another embodiment, the amount of Cannabis sativa oil in the composition is from about 10% to about 20% by weight based on the total weight of the composition. In yet another embodiment, the amount of Cannabis sativa oil in the composition is from about 20% to about 30% by weight based on the total weight of the composition. In yet another embodiment, the amount of Cannabis sativa oil in the composition is from about 30% to about 40% by weight based on the total weight of the composition. In yet another embodiment, the amount of Cannabis sativa oil in the composition is from about 40% to about 50% by weight based on the total weight of the composition. In yet another embodiment, the amount of Cannabis sativa oil in the composition is from about 50% to about 60% by weight based on the total weight of the composition. In yet another embodiment, the amount of Cannabis sativa oil in the composition is from about 60% to about 70% by weight based on the total weight of the composition. In yet another embodiment, the amount of Cannabis sativa oil in the composition is from about 70% to about 80% by weight based on the total weight of the composition. In yet another embodiment, the amount of Cannabis sativa oil in the composition is from about 80% to about 90% by weight based on the total weight of the composition.

In some embodiments, the Cannabis sativa oil is extracted from the plant. In other embodiments, the Cannabis sativa oil is extracted from the seeds. In some embodiments, the Cannabis sativa oil is Cannabis sativa seed oil. In some embodiments, the Cannabis sativa oil is machine pressed Cannabis sativa seed oil. In other embodiments, the Cannabis sativa oil is cold pressed Cannabis sativa seed oil. In some embodiments, the Cannabis sativa oil is hemp seed oil. In some embodiments, the Cannabis sativa oil is machine pressed hemp seed oil. In some embodiments, the Cannabis sativa oil is cold pressed hemp seed oil. In some embodiments, the Cannabis sativa oil is hemp oil.

In some embodiments, the composition comprises an effective amount of at least one topical analgesic from about 1% to about 30% by weight based on the total weight of the composition. In some embodiments, the amount of topical analgesic is effective for relieving pain. In some embodiments, the pain is back pain, neck pain, shoulder pain, feet pain, wrist pain, joint pain, and/or calve pain. In some embodiments, the pain is associated with arthritis, back pain, backache, strains, muscle strain, sprains and/or bruises. In some embodiments, the composition is used to treat acute pain. In some embodiments, the composition is used to treat chronic pain.

In some embodiments, the amount of topical analgesic in the composition is from about 1% to about 10% by weight based on the total weight of the composition. In another embodiment, the amount of topical analgesic in the composition is from about 5% to about 15% by weight based on the total weight of the composition. In another embodiment, the amount of topical analgesic in the composition is from about 10% to about 20% by weight based on the total weight of the composition. In another embodiment, the amount of topical analgesic in the composition is from about 15% to about 25% by weight based on the total weight of the composition. In another embodiment, the amount of topical analgesic in the composition is from about 20% to about 30% by weight based on the total weight of the composition. In another embodiment, the amount of topical analgesic in the composition is about 4% by weight based on the total weight of the composition. In another embodiment, the amount of topical analgesic in the composition is about 6% by weight based on the total weight of the composition. In another embodiment, the amount of topical analgesic in the composition is about 8% by weight based on the total weight of the composition. In another embodiment, the amount of topical analgesic in the composition is about 10% by weight based on the total weight of the composition. In another embodiment, the amount of topical analgesic in the composition is about 12% by weight based on the total weight of the composition. In yet another embodiment, the amount of topical analgesic in the composition is about 14% by weight based on the total weight of the composition. In another embodiment, the amount of topical analgesic in the composition is about 16% by weight based on the total weight of the composition. In some embodiments, the topical analgesic is trolamine salicylate. In other embodiments, the topical analgesic is menthol.

In some embodiments, the composition includes: one or more antioxidants and one or more colorants. In a further embodiment, the antioxidant is tocopheryl acetate. In some embodiments, the composition includes tocopheryl acetate from about 0.25% to about 0.75% by weight based on the total weight of the composition. In another embodiment, the amount of tocopheryl acetate is about 0.5% to about 1.0% by weight based on the total weight of the composition. In another embodiment, the amount of tocopheryl acetate is about 7.5% to about 1.25% by weight based on the total weight of the composition. In another embodiment, the amount of tocopheryl acetate is about 1.0% to about 1.5% by weight based on the total weight of the composition. In another embodiment, the amount of tocopheryl acetate is about 1.25% to about 1.75% by weight based on the total weight of the composition. In another embodiment, the amount of tocopheryl acetate is about 1.5% to about 2.0% by weight based on the total weight of the composition.

In some embodiments, the colorant is D&C Green No. 5 (Cl 61570). In other embodiments, the colorant is FD&C Blue No. 1. In other embodiments, the colorant is FD&C Yellow No. 5. In other embodiments, the colorants are FD&C Blue No. 1 and FD&C Yellow No. 5.

In some embodiments, the composition includes: a pH adjuster; and one or more preservatives. In some embodiments, the pH adjuster is sodium hydroxide. In some embodiments, the composition includes sodium hydroxide from about 0.057% to about 0.171% by weight based on the total weight of the composition. In other embodiments, the composition includes sodium hydroxide from about 0.114% to about 0.228% by weight based on the total weight of the composition. In some embodiments, the composition includes sodium hydroxide from about 0.171% to about 0.285% by weight based on the total weight of the composition. In some embodiments, the composition includes sodium hydroxide from about 0.228% to about 0.342% by weight based on the total weight of the composition. In some embodiments, the composition includes sodium hydroxide from about 0.285% to about 0.399% by weight based on the total weight of the composition. In some embodiments, the composition includes sodium hydroxide from about 0.342% to about 0.456% by weight based on the total weight of the composition.

In some embodiments, the preservative is phenoxyethanol. In some embodiments, the composition includes phenoxyethanol from about 0.225% to about 0.675% by weight based on the total weight of the composition. In other embodiments, the composition includes phenoxyethanol from about 0.45% to about 0.9% by weight based on the total weight of the composition. In other embodiments, the composition includes phenoxyethanol from about 0.675% to about 1.125% by weight based on the total weight of the composition. In other embodiments, the composition includes phenoxyethanol from about 0.9% to about 1.35% by weight based on the total weight of the composition. In other embodiments, the composition includes phenoxyethanol from about 1.125% to about 1.575% by weight based on the total weight of the composition. In other embodiments, the composition includes phenoxyethanol from about 1.35% to about 1.8% by weight based on the total weight of the composition.

In some embodiments, the preservative is caprylyl glycol. In some embodiments, the composition includes caprylyl glycol from about 0.125% to about 0.375% by weight based on the total weight of the composition. In other embodiments, the composition includes caprylyl glycol from about 0.25% to about 0.5% by weight based on the total weight of the composition. In some embodiments, the composition includes caprylyl glycol from about 0.375% to about 0.625% by weight based on the total weight of the composition. In some embodiments, the composition includes caprylyl glycol from about 0.5% to about 0.75% by weight based on the total weight of the composition. In some embodiments, the composition includes caprylyl glycol from about 0.625% to about 0.875% by weight based on the total weight of the composition. In some embodiments, the composition includes caprylyl glycol from about 0.75% to about 1.0% by weight based on the total weight of the composition.

In one exemplary embodiment, the composition includes water/aqua, ethyl hexyl stearate, butylene glycol, dimethicone, stearic acid, caprlic/capric triglyceride, glyceryl stearate, PEG-100 stearate, cetostearyl alcohol, sunflower oil, allantoin, ethylhexylglycerin, isohexadecane, sodium acrylate/sodium acryloyldimethyl taurate copolymer, acrylates/C10-30 alkyl acrylate crosspolymer, hexylene glycol, polysorbate 80, disodium EDTA, a colorant, a pH adjuster, one or more preservatives, and an antioxidant.

In another exemplary embodiment, the composition includes Arctum lappa root extract, Aloe barbadensis leaf extract, arnica montana flower extract, Boswellia carteril resin extract, Calendula officinalis extract, Camellia sinensis lead extract, camphor, carbomer, glycerin, Ilex paragiariensis leaf extract, isopropyl alcohol, isopropyl myristate, Melissa officinalis (lemon balm) extract, silica, triethanolamine and water.

In some embodiments, the composition is phthalate free. In some embodiments, the composition is paraben free. In some embodiments, the composition is vegan. In some embodiments, the composition is gluten free.

In some embodiments, the composition is a cream. In other embodiments, the composition is a gel. In further embodiments, the composition is an oil.

The topical compositions described herein can provide at least one of the following benefits: increased pain relief efficacy, increased absorption into the skin, improved texture, enhanced anti-inflammatory properties and increased moisturizing benefits.

### Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

The term “a” or “an” as used herein includes the singular and the plural, unless specifically stated otherwise. Therefore, the terms “a,” “an” or “at least one” can be used interchangeably in this application.

As used herein, the term “about” includes ±10% from the indicated values in the range. In addition, where a parameter range is provided, all integers within that range, and tenths thereof, are also provided by the invention. For example, “1% to 20%” includes 1.0%, 1.1%, 1.2%, 1.3%, 1.4% etc. up to 20%.

As used herein, the term “effective amount” refers to an amount of the active component that, when contacted or sensed, is sufficient to achieve a good level of at least one desired outcome.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments and compositions detailed are only illustrative of the invention as described more fully in the claims which follow thereafter.

The invention is illustrated by the following examples without limiting it thereby.

### EXPERIMENTAL DETAILS

### Example 1. Maximum Strength Pain Relief Cream with Cannabis Seed Extract

Another example of the composition of the present invention is the Hempvana® Pain Relief cream, which contains the following ingredients:
cannabis seed extract, trolamine salicylate (10% w/w), water/aqua, ethyl hexyl stearate, butylene glycol, dimethicone, stearic acid, caprlic/capric triglyceride, glyceryl stearate, PEG-100 stearate, cetostearyl alcohol, sunflower oil, allantoin, tocopheryl acetate, ethylhexylglycerin, isohexadecane, sodium acrylate/sodium acryloyldimethyl taurate copolymer, acrylates/C10-30 alkyl acrylate crosspolymer, caprylyl glycol, hexylene glycol, polysorbate 80, disodium EDTA, D&C Green No. 5 (Cl 61570), sodium hydroxide and phenoxyethanol.

The Hempvana® Pain Relief cream absorbs quickly and works instantly. Its unique formula includes a proven active ingredient, i.e. trolamine salicylate, plus the moisturizing benefit of cold pressed cannabis seed extract for soothing relief to back, neck, shoulders, feet, wrists, calves and more.

The Hempvana® Pain Relief cream is maximum strength, odor free, non-irritating, non-burning and non-greasy.

### Example 2. Maximum Strength Pain Relief Cream with Hemp Seed Extract

Another example of the composition of the present invention is the Hempvana® Pain Relief cream, which contains the following ingredients:
hemp seed extract, trolamine salicylate (10% w/w), water/aqua, ethyl hexyl stearate, butylene glycol, dimethicone, stearic acid, caprlic/capric triglyceride, glyceryl stearate, PEG-100 stearate, cetostearyl alcohol, sunflower oil, allantoin, tocopheryl acetate, ethylhexylglycerin, isohexadecane, sodium acrylate/sodium acryloyldimethyl taurate copolymer, acrylates/C10-30 alkyl acrylate crosspolymer, caprylyl glycol, hexylene glycol, polysorbate 80, disodium EDTA, D&C Green No. 5 (Cl 61570), sodium hydroxide and phenoxyethanol.

The Hempvana® Pain Relief cream absorbs quickly and works instantly. Its unique formula includes a proven active ingredient, i.e. trolamine salicylate, plus the moisturizing benefit of cold pressed hemp seed extract for soothing relief to back, neck, shoulders, feet, wrists, calves and more.

The Hempvana® Pain Relief cream is maximum strength, odor free, non-irritating, non-burning and non-greasy.

### Example 3. Pain Roll-on Gel

Another example of the composition of the present invention is the Hempvana® Pain Roll On gel, which contains the following ingredients:
cannabis seed extract, menthol USP (8% w/w), arctium lappa root extract, Aloe barbadensis leaf extract, arnica montana flower extract, Boswellia carteril resin extract, Calendula officinalis extract, Camellia sinensis leaf extract, camphor, carbomer, FD&C Blue No. 1, FD&C Yellow No. 5, glycerin, Ilex paragiariensis leaf extract, isopropyl alcohol, isopropyl myristate, Melissa officinalis (lemon balm) extract, silica, tocopheryl acetate, triethanolamine, and water.

The Hempvana® Pain Roll On gel uses a proven active ingredient, i.e. menthol USP, plus the moisturizing benefit of cannabis seed extract. The Hempvana® Pain Roll On gel may be used for arthritis, back, shoulder, joint and muscle pain.

### Example 4a. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 68.1655% | 100% | 68.1655% | Solvent |
| Carbopol Ultrez | Acrylates/C10-30 Alkyl | 0.57% | 100% | 0.57% | Viscosity Increasing Agents- |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents- |
| | | | | | Miscellaneous |
| Allantoin USP | Allantoin | 0.1% | 100% | 0.1% | Skin-Conditioning Agents- |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents- |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents- |
| | | | | | Emollient |
| Sunflower Oil | Helianthus Annuus | 1% | 100% | 1% | Skin-Conditioning Agents- |
| | (Sunflower) Seed Oil | | | | Emollient |
| Botanester GC/ | Caprylic/Capric | 2% | 100% | 2% | Skin-Conditioning Agents- |
| Liponate GC-K | Triglyceride | | | | Occlusive |
| Xiameter PMX- | Dimethicone | 2.5% | 100% | 2.5% | Skin-Conditioning Agents- |
| 200 Silicone Fluid | | | | | Occlusive |
| 100 CS | | | | | |
| Ritapro 165 | Glyceryl Stearate | 3% | 50% | 1.5% | Skin-Conditioning Agents- |
| | | | | | Emollient |
| | PEG-100 Stearate | | 50% | 1.5% | Surfactants-Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants-Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents- |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents- |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents- |
| | | | | | Emollient |
| | Polysorbate 80 | | 7.5% | 0.075% | Surfactants-Emulsifying |
| | | | | | Agents |
| | Sorbitan Oleate* | | 2.5% | 0.025% | Surfactants-Emulsifying |
| | | | | | Agents |

### Example 4b. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 67.1655% | 100% | 67.1655% | Solvent |
| Carbopol Ultrez | Acrylates/C10-30 Alkyl | 0.57% | 100% | 0.57% | Viscosity Increasing Agents- |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents- |
| | | | | | Miscellaneous |
| Allantoin USP | Allantoin | 0.1% | 100% | 0.1% | Skin-Conditioning Agents- |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents- |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents- |
| | | | | | Emollient |
| Sunflower Oil | Helianthus Annuus | 1% | 100% | 1% | Skin-Conditioning Agents- |
| | (Sunflower) Seed Oil | | | | Emollient |
| Botanester GC/ | Caprylic/Capric | 2% | 100% | 2% | Skin-Conditioning Agents- |
| Liponate GC-K | Triglyceride | | | | Occlusive |
| Xiameter PMX- | Dimethicone | 2.5% | 100% | 2.5% | Skin-Conditioning Agents- |
| 200 Silicone Fluid | | | | | Occlusive |
| 100 CS | | | | | |
| Ritapro 165 | Glyceryl Stearate | 3% | 50% | 1.5% | Skin-Conditioning Agents- |
| | | | | | Emollient |
| | PEG-100 Stearate | | 50% | 1.5% | Surfactants-Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants-Emulsifying |
| Stearic Acid/ | | | | | Agents |
| Ritastearic | | | | | |
| Caribpure Hemp | Cannabis Sativa (Hemp) | 1.5% | 100% | 1.5% | Skin-Conditioning Agents- |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents- |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents- |
| | | | | | Emollient |
| | Polysorbate 80 | | 7.5% | 0.075% | Surfactants-Emulsifying |
| | | | | | Agents |
| | Sorbitan Oleate* | | 2.5% | 0.025% | Surfactants-Emulsifying |
| | | | | | Agents |

### Example 4c. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 66.1655% | 100% | 66.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4d. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 63.6655% | 100% | 63.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 5% | 100% | 5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4e. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 58.6655% | 100% | 58.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 10% | 100% | 10% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4f. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 63.1655% | 100% | 63.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4g. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 62.1655% | 100% | 62.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Saliva (Hemp) | 1.5% | 100% | 1.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4h. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 61.1655% | 100% | 61.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4i. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 58.6655% | 100% | 58.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 5% | 100% | 5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4j. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 53.6655% | 100% | 53.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 10% | 100% | 10% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4k. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 58.1655% | 100% | 58.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 15% | 100% | 15% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4l. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 57.1655% | 100% | 57.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 1.5% | 100% | 1.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 15% | 100% | 15% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4m. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 56.1655% | 100% | 56.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 15% | 100% | 15% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4n. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 53.1655% | 100% | 53.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 20% | 100% | 20% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4o. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 52.1655% | 100% | 52.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 1.5% | 100% | 1.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 20% | 100% | 20% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4p. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 51.1655% | 100% | 51.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 0.5% | 100% | 0.5% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 20% | 100% | 20% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4q. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 67.6655% | 100% | 67.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4r. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 66.6655% | 100% | 66.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 1.5% | 100% | 1.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4s. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 65.6655% | 100% | 65.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4t. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 63.1655% | 100% | 63.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 5% | 100% | 5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4u. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 58.1655% | 100% | 58.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 10% | 100% | 10% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 5% | 100% | 5% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4v. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 62.6655% | 100% | 62.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4w. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 61.6655% | 100% | 61.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 1.5% | 100% | 1.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4x. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 60.6655% | 100% | 60.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4y. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 58.1655% | 100% | 58.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 5% | 100% | 5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4z. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 53.1655% | 100% | 53.1655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 10% | 100% | 10% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 10% | 100% | 10% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4aa. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 57.6655% | 100% | 57.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 0.5% | 100% | 0.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 15% | 100% | 15% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4bb. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 56.6655% | 100% | 56.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 1.5% | 100% | 1.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 15% | 100% | 15% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4cc. Composition Formulation

| | | | | Finished | |
|---|---|---|---|---|---|
| | | | Ingredient | Formula | |
| Trade Name | INCI Nomenclature | Formula % | Activity % | Conc. | Function |
| Deionized Water | Water | 55.6655% | 100% | 55.6655% | Solvent |
| Carbopol Ultrez | Acrylates/C10-30 Alkyl | 0.57% | 100% | 0.57% | Viscosity Increasing Agents - |
| 20 | Acrylate Crosspolymer | | | | Aqueous |
| Dissolvine NA2-P | Disodium EDTA | 0.05% | 100% | 0.05% | Chelating Agents |
| Butylene Glycol | Butylene Glycol | 5% | 100% | 5% | Skin-Conditioning Agents - |
| | | | | | Miscellaneous |
| Allantoin USP | Allantoin | 0.1% | 100% | 0.1% | Skin-Conditioning Agents - |
| | | | | | Miscellaneous |
| Botanistat PF-64 | Phenoxyethanol | 1% | 45% | 0.45% | Preservatives |
| | Caprylyl Glycol | | 25% | 0.25% | Preservatives |
| | Ethylhexylglycerin | | 15% | 0.15% | Skin-Conditioning Agents - |
| | | | | | Miscellaneous |
| | Hexylene Glycol | | 15% | 0.15% | Solvents |
| Lexfeel EHS | Ethylhexyl Stearate | 6% | 100% | 6% | Skin-Conditioning Agents - |
| | | | | | Emollient |
| Sunflower Oil | Helianthus Annuus | 1% | 100% | 1% | Skin-Conditioning Agents - |
| | (Sunflower) Seed Oil | | | | Emollient |
| Botanester GC/ | Caprylic/Capric | 2% | 100% | 2% | Skin-Conditioning Agents - |
| Liponate GC-K | Triglyceride | | | | Occlusive |
| Xiameter PMX- | Dimethicone | 2.5% | 100% | 2.5% | Skin-Conditioning Agents - |
| 200 Silicone Fluid | | | | | Occlusive |
| 100 CS | | | | | |
| Ritapro 165 | Glyceryl Stearate | 3% | 50% | 1.5% | Skin-Conditioning Agents - |
| | | | | | Emollient |
| | PEG-100 Stearate | | 50% | 1.5% | Surfactants - Cleansing |
| | | | | | Agents |
| Cetearyl Alcohol | Cetearyl Alcohol | 1% | 100% | 1% | Emulsion Stabilizers |
| Triple Pressed | Stearic Acid | 2.5% | 100% | 2.5% | Surfactants- Emulsifying |
| Stearic Acid/ | | | | | Agents |
| Ritastearic | | | | | |
| Caribpure Hemp | Cannabis Sativa (Hemp) | 2.5% | 100% | 2.5% | Skin-Conditioning Agents - |
| Seed Oil Refined | Seed Oil | | | | Miscellaneous |
| dl-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide Solid | | | | | |
| Green #5 (CI | Green 5 | 0.0005% | 100% | 0.0005% | Colorants |
| 61570) | | | | | |
| TEA-Salicylate | TEA-Salicylate | 15% | 100% | 15% | Light Stabilizers |
| Solid | | | | | |
| Simulgel EG | Sodium | 1% | 37.5% | 0.375% | Viscosity Increasing Agents - |
| | Acrylate/Sodium | | | | Aqueous |
| | Acryloyldimethyl | | | | |
| | Taurate Copolymer | | | | |
| | Water | | 30% | 0.3% | Solvent |
| | Isohexadecane | | 22.5% | 0.225% | Skin-Conditioning Agents - |
| | | | | | Emollient |
| | Polysorbate 80 | | 7.5% | 0.075% | Surfactants - Emulsifying |
| | | | | | Agents |
| | Sorbitan Oleate* | | 2.5% | 0.025% | Surfactants - Emulsifying |
| | | | | | Agents |

### Example 4dd. Composition Formulation

| | | | | Finished | |
|---|---|---|---|---|---|
| | INCI | | Ingredient | Formula | |
| Trade Name | Nomenclature | Formula % | Activity % | Conc. | Function |
| Deionized | Water | 52.6655% | 100% | 52.6655% | Solvent |
| Water | | | | | |
| Carbopol | Acrylates/ | 0.57% | 100% | 0.57% | Viscosity |
| Ultrez 20 | C10-30 Alkyl | | | | Increasing |
| | Acrylate | | | | Agents - |
| | Crosspolymer | | | | Aqueous |
| Dissolvine | Disodium EDTA | 0.05% | 100% | 0.05% | Chelating |
| NA2-P | | | | | Agents |
| Butylene | Butylene Glycol | 5% | 100% | 5% | Skin- |
| Glycol | | | | | Conditioning |
| | | | | | Agents - |
| | | | | | Miscellaneous |
| Allantoin | Allantoin | 0.1% | 100% | 0.1% | Skin- |
| USP | | | | | Conditioning |
| | | | | | Agents - |
| | | | | | Miscellaneous |
| Botanistat | Phenoxyethanol | 1% | 45% | 0.45% | Preservatives |
| PF-64 | Caprylyl Glycol | | 25% | 0.25% | Preservatives |
| | Ethylhexylglycerin | | 15% | 0.15% | Skin- |
| | | | | | Conditioning |
| | | | | | Agents - |
| | | | | | Miscellaneous |
| | Hexylene Glycol | | 15% | 0.15% | Solvents |
| Lexfeel EHS | Ethylhexyl Stearate | 6% | 100% | 6% | Skin- |
| | | | | | Conditioning |
| | | | | | Agents - |
| | | | | | Emollient |
| Sunflower | Helianthus Annuus | 1% | 100% | 1% | Skin- |
| Oil | (Sunflower) Seed Oil | | | | Conditioning |
| | | | | | Agents - |
| | | | | | Emollient |
| Botanester | Caprylic/Capric | 2% | 100% | 2% | Skin- |
| GC/Liponate | Triglyceride | | | | Conditioning |
| GC-K | | | | | Agents - |
| | | | | | Occlusive |
| Xiameter | Dimethicone | 2.5% | 100% | 2.5% | Skin- |
| PMX-200 | | | | | Conditioning |
| Silicone | | | | | Agents - |
| Fluid 100 CS | | | | | Occlusive |
| Ritapro 165 | Glyceryl Stearate | 3% | 50% | 1.5% | Skin- |
| | | | | | Conditioning |
| | | | | | Agents - |
| | | | | | Emollient |
| | PEG-100 Stearate | | 50% | 1.5% | Surfactants - |
| | | | | | Cleansing |
| | | | | | Agents |
| Cetearyl | Cetearyl Alcohol | 1% | 100% | 1% | Emulsion |
| Alcohol | | | | | Stabilizers |
| Triple | Stearic Acid | 2.5% | 100% | 2.5% | Surfactants- |
| Pressed | | | | | Emulsifying |
| Stearic | | | | | Agents |
| Acid/ | | | | | |
| Ritastearic | | | | | |
| Caribpure | Cannabis Sativa | 0.5% | 100% | 0.5% | Skin- |
| Hemp Seed | (Hemp) Seed Oil | | | | Conditioning |
| Oil Refined | | | | | Agents - |
| | | | | | Miscellaneous |
| d1-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide | | | | | |
| Solid | | | | | |
| Green #5 | Green 5 | 0.0005% | 100% | 0.0005% | Colorants |
| (CI 61570) | | | | | |
| TEA- | TEA-Salicylate | 20% | 100% | 20% | Light |
| Salicylate | | | | | Stabilizers |
| Solid | | | | | |
| Simulgel EG | Sodium | 1% | 37.5% | 0.375% | Viscosity |
| | Acrylate/Sodium | | | | Increasing |
| | Acryloyldimethyl | | | | Agents - |
| | Taurate Copolymer | | | | Aqueous |
| | Water | | 30% | 0.3% | Solvent |
| | Isohexadecane | | 22.5% | 0.225% | Skin- |
| | | | | | Conditioning |
| | | | | | Agents - |
| | | | | | Emollient |
| | Polysorbate 80 | | 7.5% | 0.075% | Surfactants - |
| | | | | | Emulsifying |
| | | | | | Agents |
| | Sorbitan Oleate* | | 2.5% | 0.025% | Surfactants - |
| | | | | | Emulsifying |
| | | | | | Agents |

### Example 4ee. Composition Formulation

| | | | | Finished | |
|---|---|---|---|---|---|
| | INCI | | Ingredient | Formula | |
| Trade Name | Nomenclature | Formula % | Activity % | Conc. | Function |
| Deionized | Water | 51.6655% | 100% | 51.6655% | Solvent |
| Water | | | | | |
| Carbopol | Acrylates/ | 0.57% | 100% | 0.57% | Viscosity |
| Ultrez 20 | C10-30 Alkyl | | | | Increasing |
| | Acrylate | | | | Agents - |
| | Crosspolymer | | | | Aqueous |
| Dissolvine | Disodium EDTA | 0.05% | 100% | 0.05% | Chelating |
| NA2-P | | | | | Agents |
| Butylene | Butylene Glycol | 5% | 100% | 5% | Skin- |
| Glycol | | | | | Conditioning |
| | | | | | Agents - |
| | | | | | Miscellaneous |
| Allantoin | Allantoin | 0.1% | 100% | 0.1% | Skin- |
| USP | | | | | Conditioning |
| | | | | | Agents - |
| | | | | | Miscellaneous |
| Botanistat | Phenoxyethanol | 1% | 45% | 0.45% | Preservatives |
| PF-64 | Caprylyl Glycol | | 25% | 0.25% | Preservatives |
| | Ethylhexylglycerin | | 15% | 0.15% | Skin- |
| | | | | | Conditioning |
| | | | | | Agents - |
| | | | | | Miscellaneous |
| | Hexylene Glycol | | 15% | 0.15% | Solvents |
| Lexfeel EHS | Ethylhexyl Stearate | 6% | 100% | 6% | Skin- |
| | | | | | Conditioning |
| | | | | | Agents - |
| | | | | | Emollient |
| Sunflower | Helianthus Annuus | 1% | 100% | 1% | Skin- |
| Oil | (Sunflower) Seed Oil | | | | Conditioning |
| | | | | | Agents - |
| | | | | | Emollient |
| Botanester | Caprylic/Capric | 2% | 100% | 2% | Skin- |
| GC/Liponate | Triglyceride | | | | Conditioning |
| GC-K | | | | | Agents - |
| | | | | | Occlusive |
| Xiameter | Dimethicone | 2.5% | 100% | 2.5% | Skin- |
| PMX-200 | | | | | Conditioning |
| Silicone | | | | | Agents - |
| Fluid 100 CS | | | | | Occlusive |
| Ritapro 165 | Glyceryl Stearate | 3% | 50% | 1.5% | Skin- |
| | | | | | Conditioning |
| | | | | | Agents - |
| | | | | | Emollient |
| | PEG-100 Stearate | | 50% | 1.5% | Surfactants - |
| | | | | | Cleansing |
| | | | | | Agents |
| Cetearyl | Cetearyl Alcohol | 1% | 100% | 1% | Emulsion |
| Alcohol | | | | | Stabilizers |
| Triple | Stearic Acid | 2.5% | 100% | 2.5% | Surfactants- |
| Pressed | | | | | Emulsifying |
| Stearic | | | | | Agents |
| Acid/ | | | | | |
| Ritastearic | | | | | |
| Caribpure | Cannabis Sativa | 1.5% | 100% | 1.5% | Skin- |
| Hemp Seed | (Hemp) Seed Oil | | | | Conditioning |
| Oil Refined | | | | | Agents - |
| | | | | | Miscellaneous |
| d1-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide | | | | | |
| Solid | | | | | |
| Green #5 | Green 5 | 0.0005% | 100% | 0.0005% | Colorants |
| (CI 61570) | | | | | |
| TEA- | TEA-Salicylate | 20% | 100% | 20% | Light |
| Salicylate | | | | | Stabilizers |
| Solid | | | | | |
| Simulgel EG | Sodium | 1% | 37.5% | 0.375% | Viscosity |
| | Acrylate/Sodium | | | | Increasing |
| | Acryloyldimethyl | | | | Agents - |
| | Taurate Copolymer | | | | Aqueous |
| | Water | | 30% | 0.3% | Solvent |
| | Isohexadecane | | 22.5% | 0.225% | Skin- |
| | | | | | Conditioning |
| | | | | | Agents - |
| | | | | | Emollient |
| | Polysorbate 80 | | 7.5% | 0.075% | Surfactants - |
| | | | | | Emulsifying |
| | | | | | Agents |
| | Sorbitan Oleate* | | 2.5% | 0.025% | Surfactants - |
| | | | | | Emulsifying |
| | | | | | Agents |

### Example 4ff. Composition Formulation

| | | | | Finished | |
|---|---|---|---|---|---|
| | INCI | | Ingredient | Formula | |
| Trade Name | Nomenclature | Formula % | Activity % | Conc. | Function |
| Deionized | Water | 50.6655% | 100% | 50.6655% | Solvent |
| Water | | | | | |
| Carbopol | Acrylates/ | 0.57% | 100% | 0.57% | Viscosity |
| Ultrez 20 | C10-30 Alkyl | | | | Increasing |
| | Acrylate | | | | Agents - |
| | Crosspolymer | | | | Aqueous |
| Dissolvine | Disodium EDTA | 0.05% | 100% | 0.05% | Chelating |
| NA2-P | | | | | Agents |
| Butylene | Butylene Glycol | 5% | 100% | 5% | Skin- |
| Glycol | | | | | Conditioning |
| | | | | | Agents - |
| | | | | | Miscellaneous |
| Allantoin | Allantoin | 0.1% | 100% | 0.1% | Skin- |
| USP | | | | | Conditioning |
| | | | | | Agents - |
| | | | | | Miscellaneous |
| Botanistat | Phenoxyethanol | 1% | 45% | 0.45% | Preservatives |
| PF-64 | Caprylyl Glycol | | 25% | 0.25% | Preservatives |
| | Ethylhexylglycerin | | 15% | 0.15% | Skin- |
| | | | | | Conditioning |
| | | | | | Agents - |
| | | | | | Miscellaneous |
| | Hexylene Glycol | | 15% | 0.15% | Solvents |
| Lexfeel EHS | Ethylhexyl Stearate | 6% | 100% | 6% | Skin- |
| | | | | | Conditioning |
| | | | | | Agents - |
| | | | | | Emollient |
| Sunflower | Helianthus Annuus | 1% | 100% | 1% | Skin |
| Oil | (Sunflower) Seed Oil | | | | Conditioning |
| | | | | | Agents - |
| | | | | | Emollient |
| Botanester | Caprylic/Capric | 2% | 100% | 2% | Skin- |
| GC/Liponate | Triglyceride | | | | Conditioning |
| GC-K | | | | | Agents - |
| | | | | | Occlusive |
| Xiameter | Dimethicone | 2.5% | 100% | 2.5% | Skin- |
| PMX-200 | | | | | Conditioning |
| Silicone | | | | | Agents - |
| Fluid 100 CS | | | | | Occlusive |
| Ritapro 165 | Glyceryl Stearate | 3% | 50% | 1.5% | Skin- |
| | | | | | Conditioning |
| | | | | | Agents - |
| | | | | | Emollient |
| | PEG-100 Stearate | | 50% | 1.5% | Surfactants - |
| | | | | | Cleansing |
| | | | | | Agents |
| Cetearyl | Cetearyl Alcohol | 1% | 100% | 1% | Emulsion |
| Alcohol | | | | | Stabilizers |
| Triple | Stearic Acid | 2.5% | 100% | 2.5% | Surfactants- |
| Pressed | | | | | Emulsifying |
| Stearic | | | | | Agents |
| Acid/ | | | | | |
| Ritastearic | | | | | |
| Caribpure | Cannabis Sativa | 2.5% | 100% | 2.5% | Skin- |
| Hemp Seed | (Hemp) Seed Oil | | | | Conditioning |
| Oil Refined | | | | | Agents - |
| | | | | | Miscellaneous |
| d1-alpha | Tocopheryl Acetate | 1% | 100% | 1% | Antioxidants |
| Tocopheryl | | | | | |
| Acetate | | | | | |
| Sodium | Sodium Hydroxide | 0.114% | 100% | 0.114% | pH Adjusters |
| Hydroxide | | | | | |
| Solid | | | | | |
| Green #5 | Green 5 | 0.0005% | 100% | 0.0005% | Colorants |
| (CI 61570) | | | | | |
| TEA- | TEA-Salicylate | 20% | 100% | 20% | Light |
| Salicylate | | | | | Stabilizers |
| Solid | | | | | |
| Simulgel EG | Sodium | 1% | 37.5% | 0.375% | Viscosity |
| | Acrylate/Sodium | | | | Increasing |
| | Acryloyldimethyl | | | | Agents - |
| | Taurate Copolymer | | | | Aqueous |
| | Water | | 30% | 0.3% | Solvent |
| | Isohexadecane | | 22.5% | 0.225% | Skin- |
| | | | | | Conditioning |
| | | | | | Agents - |
| | | | | | Emollient |
| | Polysorbate 80 | | 7.5% | 0.075% | Surfactants - |
| | | | | | Emulsifying |
| | | | | | Agents |
| | Sorbitan Oleate* | | 2.5% | 0.025% | Surfactants |
| | | | | | Emulsifying |
| | | | | | Agents |

Discussion

Cannabis is a genus of plants belonging to the Cannabaceae family. The cannabis plant is widely studied and has many known beneficial properties. Species of cannabis include Cannabis sativa, Cannabis indica, and Cannabis ruderalis. Hemp is a strain of Cannabis sativa that is commonly used for industrial purposes.

Oil can be extracted from Cannabis sativa. Specifically, oil can be extracted from Cannabis sativa seeds. Cannabis sativa oil has many known health benefits. The moisturizing properties of Cannabis sativa oil make it beneficial for use on the skin. There are several known methods of extracting Cannabis seed oil, one of which is to machine press the Cannabis sativa seeds. Another way is to cold-press the seeds from Cannabis sativa. Evidence suggests that cannabis is safe and can be used as an inexpensive alternative to other known substances with similar properties.

Cannabis sativa oil is known to contain essential polyunsaturated fatty acids, including omega-3s and omega-6s. More specifically, Cannabis sativa oil contains gamma linolenic acid, a type of omega-6 fatty acid. Polyunsaturated fatty acids are known to help with certain health issues, such as inflammation and skin conditions. Specifically, it is known that a 3:1 ratio of omega-6 to omega-3 can reduce inflammation and alleviate pain. Cannabis sativa oil has a balanced 3:1 ratio of omega-6 to omega-3 essential fatty acids.

When a skin moisturizing composition uses a topical analgesic in combination with cannabis seed oil, the composition as a whole has various improvements and additional advantages over the prior art. The addition of the Cannabis sativa oil enhances the pain-relieving properties of the topical analgesic. The combination of the topical analgesic and the Cannabis sativa oil increases the pain-relieving efficacy of the composition. The addition of Cannabis sativa oil to the composition also provides an enhanced texture, making it more desirable for use on skin. Further, the addition of Cannabis sativa oil also results in the composition having enhanced moisturizing benefits. Additionally, the combination of Cannabis sativa oil and a topical analgesic result in increased anti-inflammatory qualities in the composition. Also, the combination has enhanced skin absorption properties. These qualities are advantageous for topical pain-relieving compositions. The formulations using both Cannabis sativa oil and a topical analgesic have these enhanced properties which are alternatives and improvements to the prior art compositions.

1. A topical skin moisturizing composition comprising:
Cannabis sativa oil in an effective amount from about 1% to about 90%; and
at least one topical analgesic in an effective amount from about 1% to about 20% by weight based on the total weight of the composition.

2. The composition of claim 1, wherein the Cannabis sativa oil is Cannabis sativa seed oil.

3. The composition of claim 1, further comprising:
one or more antioxidants; and
one or more colorants.

4. The composition of claim 3, wherein the antioxidant is tocopheryl acetate.

5. The composition of claim 3, wherein the topical analgesic is trolamine salicylate.

6. The composition of claim 5, further comprising:
a pH adjuster; and
one or more preservatives.

7. The composition of claim 6, wherein the pH adjuster is sodium hydroxide.

8. The composition of claim 6, wherein in the one or more preservatives are phenoxyethanol and caprylyl glycol.

9. The composition of claim 6, wherein the colorant is D&C Green No. 5 (Cl 61570).

10. The composition of claim 1, wherein the amount of topical analgesic in the composition is effective for relieving back pain, neck pain, shoulder pain, feet pain, wrist pain, joint pain, and/or calve pain.

11. The composition of claim 1, wherein the amount of topical analgesic in the composition is effective for relieving pain associated with arthritis, back pain, backache, strains, muscle strain, sprains and/or bruises.

12. The composition of claim 1, wherein the composition is a cream.

13. The composition of claim 1, wherein the composition is phthalate free, paraben free, vegan and/or gluten free.

14. The composition of claim 1, wherein the composition comprises:
from about 1% to about 20% trolamine salicylate, from about 1% to about 20% Cannabis sativa oil, from about 3% to about 12% ethyl hexyl stearate, from about 2.5% to about 10% butylene glycol, from about 1.25% to about 5% dimethicone, from about 1.25% to about 5% stearic acid, from about 1% to about 4% caprlic/capric triglyceride, from about 0.75% to about 3% glyceryl stearate, from about 0.75% to about 3% PEG-100 stearate, from about 0.5% to about 2% cetostearyl alcohol, from about 0.5% to about 2% sunflower oil, from about 0.1% to about 0.2% allantoin, from about 0.5% to about 1% tocopheryl acetate, from about 0.15% to about 0.3% ethylhexylglycerin, from about 0.225% to about 0.45% isohexadecane, from about 0.375% to about 0.75% sodium acrylate/sodium acryloyldimethyl taurate copolymer, from about 0.57% to about 1.14% acrylates/C10-30 alkyl acrylate crosspolymer, from about 0.25% to about 0.5% caprylyl glycol, from about 0.15% to about 0.3% hexylene glycol, from about 0.075% to about 0.15% polysorbate 80, from about 0.05% to about 0.1% disodium EDTA, from about 0.0005% to about 0.001% D&C Green No. 5 (Cl 61570), from about 0.114% to about 0.228% sodium hydroxide, from about 0.025% to about 0.05% sorbitan oleat, and from about 0.45% to about 0.9% phenoxyethanol by weight based on the total weight of the composition, and water in an amount to make up 100%.

15. The composition of claim 3, wherein the topical analgesic is menthol USP.

16. The composition of claim 15, comprises from about 1 to about 20% menthol USP, Arctum lappa root extract, Aloe barbadensis leaf extract, Arnica montana flower extract, Boswellia carteril resin extract, Calendula officinalis extract, Camellia sinensis lead extract, camphor, carbomer, glycerin, Ilex paragiariensis leaf extract, isopropyl alcohol, isopropyl myristate, Melissa officinalis (lemon balm) extract, silica, triethanolamine and water.

17. The composition of claim 16, wherein the one or more colorants are FD&C Blue No. 1 and FD&C Yellow No. 5.

18. The composition of claim 16, wherein the composition is a gel.

19. The composition of claim 16, wherein the composition is phthalate free, paraben free, vegan and/or gluten free.

20. A method for the treatment of pain in a subject comprising:
a. topically applying to the skin, a skin moisturizing composition consisting of:
i. Cannabis sativa oil in an effective amount from about 1% to about 20%;
ii. at least one topical analgesic in an effective amount from about 1% to about 20% by weight based on the total weight of the composition;
iii. one or more antioxidants; and
iv. one or more colorants.
